(19) United States
(12) Patent Application Publication
(10) Pub. No.: US 2005/0267515 A1
(43) Pub. Date: Dec. 1, 2005

(54) Embolus blood clot filter

(75) Inventors: Vincent Oliva, Mont-Royal (CA); Gilles Soulez, Outremont (CA)

Correspondence Address: OGILVY RENAULT LLP, 1981 MCGILL COLLEGE AVENUE, SUITE 1600, MONTREAL, QC, H3A2Y3, CA

(73) Assignee: Vascular Interventional Technologies Inc.

(21) Appl. No.: 11/139,529

(22) Filed: May 31, 2005

Related U.S. Application Data
(63) Continuation of application No. PCT/CA03/01864, filed on Nov. 28, 2003.
(60) Provisional application No. 60/429,545, filed on Nov. 29, 2002.

(51) Int. Cl. A61M 29/00

(52) U.S. Cl. 606/200

(57) ABSTRACT

A blood clot filter (10) comprises a number of diverging legs (26) extending from a common central hub (16) to define a blood clot reservoir. Adjacent legs (26) share a common root (18) from which branches off at least two main branches (24). Each main branch (24) connects at a distal end to an adjacent main branch (24) to form a plurality of interconnected anchoring members (30a, 30b) about an open upstream end of the blood clot reservoir.

### RELATED APPLICATIONS

This is a continuation of International Patent Application No. PCT/CA2003/001864 filed Nov. 28, 2003, which claims benefit of U.S. Provisional Patent Application No. 60/429,545 filed on Nov. 29, 2002. The contents of both of which are herein incorporated by reference.

### BACKGROUND OF THE INVENTION

1. Field of the Invention

The present invention relates to filters and, more particularly, to a blood clot filter which is particularly suited for filtering emboli from blood circulating through a blood vessel.

2. Description of the Prior Art

It is well known to introduce a filter in the inferior vena cava of a patient to prevent pulmonary embolism. Most presently available blood clot filters are permanently implanted in the inferior vena cava and remain there for the duration of the patient's life. One drawback associated with permanent filters resides in the fact that permanent filters can become clogged and cause obstruction of the inferior vena cava, thereby resulting in chronic swelling of the lower extremities. Efforts have thus been made to develop removable or retrievable vena cava filters.

In an attempt to provide retrievable filters, it has been proposed to provide radially collapsible filters with retrieving aids, such as hooks, by which the filters can be caught and then percutaneously withdrawn from the patient's blood vessel through a catheter. However, in practice, particularly where a filter of this type is engaged on a relatively long portion of the length thereof with the inner wall of a blood vessel, such as in the case of the filter disclosed in U.S. Pat. No. 6,059,825 issued on May 9, 2002 to Hobbs et al., it has been found that such “retrievable” filters often become securely embedded in the wall of the blood vessel as their wall engaging portions become endothelialized by the vessel wall, whereby any subsequent attempt to percutaneously remove the filters results in serious risks of injury to the blood vessel or inability to remove these filters. On the other hand, where the filter is of a generally conical shape and anchored to the vessel wall only at a distal end thereof, as for instance disclosed in U.S. Pat. No. 5,324,304 issued on Jun. 28, 1994 to Rasmussen, it becomes difficult to centrally position and maintain the filter in axial alignment with the blood vessel. This might impede the filter's ability to catch clots and renders the recovery of the filter more difficult.

The need for centrally positioning and maintaining the filter in axial alignment with the blood vessel and the need for minimizing the integration of the filter with the wall of the blood vessel to permit easy retrieval of the filter have been heretofore difficult to satisfy simultaneously.

In view of the foregoing, there is thus a need for a new blood clot filter having self-centering and anti-tilting features while still being not prone to rapid integration. It has also been found that there is a need for a new blood clot filter having added clots holding capacity and which is of a more sturdy construction.

### SUMMARY OF THE INVENTION

It is therefore an aim of the present invention to provide a new blood clot filter having self-centering feature while still being easily retrievable for a relatively long period of time after having been implanted into a patient's blood vessel.

It is also an aim of the present invention to provide a blood clot filter that can still be easily removed from a blood vessel without important risk of injury to the vessel wall even after an extended period of implantation.

It is a further aim of the present invention to provide a blood clot filter having improved stability once implanted into a blood vessel of a patient.

It is a still further aim of the present invention to provide a blood clot filter that is of a more sturdy construction.

It is a still further aim of the present invention to provide a blood clot filter having added clot holding capacity.

Therefore, in accordance with the present invention, there is provided a filter positionable into a blood vessel for filtering blood clots from a stream of blood, the filter comprising a central hub, a plurality of interconnected legs extending from said central hub and arranged about a central longitudinal axis of the filter to form a filter basket having an open end, wherein each leg is provided at a distal end thereof opposite said hub with an anchoring limb adapted to anchor the filter basket to the blood vessel, wherein said anchoring limbs are distributed about said open end of said filter basket and extend at different distances from said central hub to provide at least two anchoring levels.

In accordance with another general aspect of the present invention, there is provided a blood clot filter comprising a set of diverging primary branches extending from a common central hub, each primary branch bifurcating into a pair of main branches, wherein each main branch connects at a distal end to an adjacent main branch originating from a different primary branch to form a series of interconnected diverging anchoring members.

In accordance with a further general aspect of the present invention, there is provided a filter positionable into a blood vessel for trapping blood clots, comprising a number of legs extending from a common central hub to define a blood clot reservoir, wherein adjacent legs share a common root from which branches off at least two main branches, each main branches connecting at a distal end to an adjacent main branch to form a plurality of interconnected anchoring members about an open upstream end of said blood clot reservoir.

### BRIEF DESCRIPTION OF THE DRAWINGS

Having thus generally described the nature of the invention, reference will now be made to the accompanying drawings, showing by way of illustration a preferred embodiment thereof, and in which:

FIG. 1 is a schematic side elevation view, in section, of a blood vessel in which a self-expending, retrievable blood clot filter has been implanted, the filter being shown in a partly expanded state, in accordance with a first embodiment of the present invention;

FIG. 2 is a perspective view of the filter shown in a fully expanded state; and

FIG. 3 is a top plan view of the filter shown in FIG. 2.

### DESCRIPTION OF THE PREFERRED EMBODIMENTS

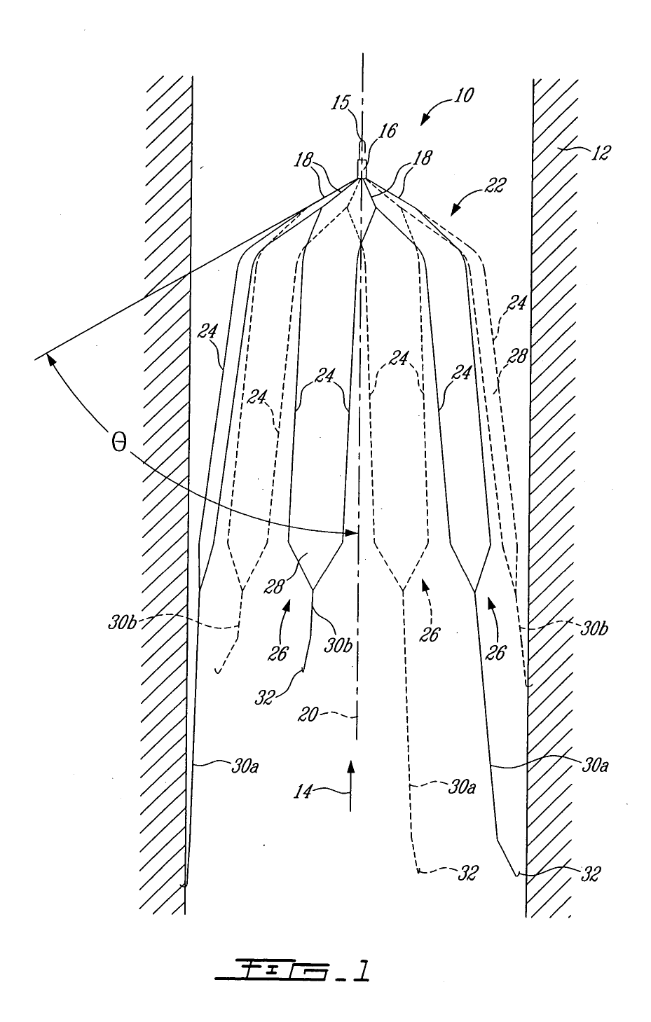

Referring now to FIG. 1, a self-centering, self-expandable blood clot filter 10 is shown anchored at two longitudinally spaced-apart locations in a blood vessel 12 (e.g. the inferior vena cava) for filtering blood clots from a stream of blood flowing in the direction indicated by arrow 14. The filter 10 is preferably placed in the vessel 12 via a delivery catheter (e.g. a 6 French Catheter) inserted through a puncture in the vessel 12. When in the catheter, the filter 10 is in a radially contracted state. Upon release from the distal end of the catheter, the filter 10 radially expands to its deployed state for securely engaging the inner wall of the vessel 12, thereby ensuring centering of the filter 10 in the vessel 12, as shown in FIG. 1. The filter 10 is preferably made out of a shape memory material or a temperature responsive material. According to a preferred embodiment of the present invention, the filter 10 is formed of Nitinol, an alloy of titanium and nickel. In this way, the filter 10 can be easily compressed and inserted within a catheter when exposed to temperature below a predetermined transition temperature, but at temperatures at or above the transition temperature, the filter 10 expands and becomes relatively rigid.

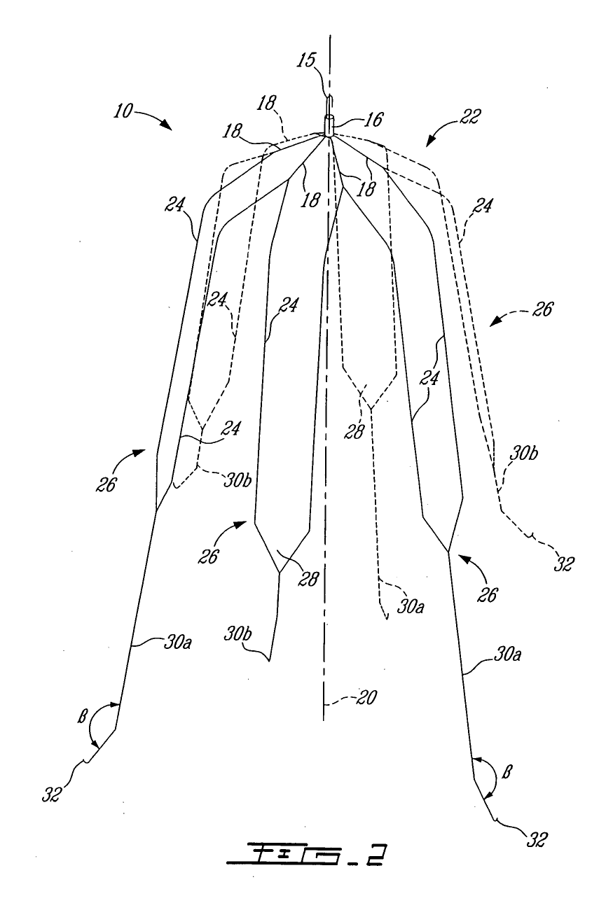

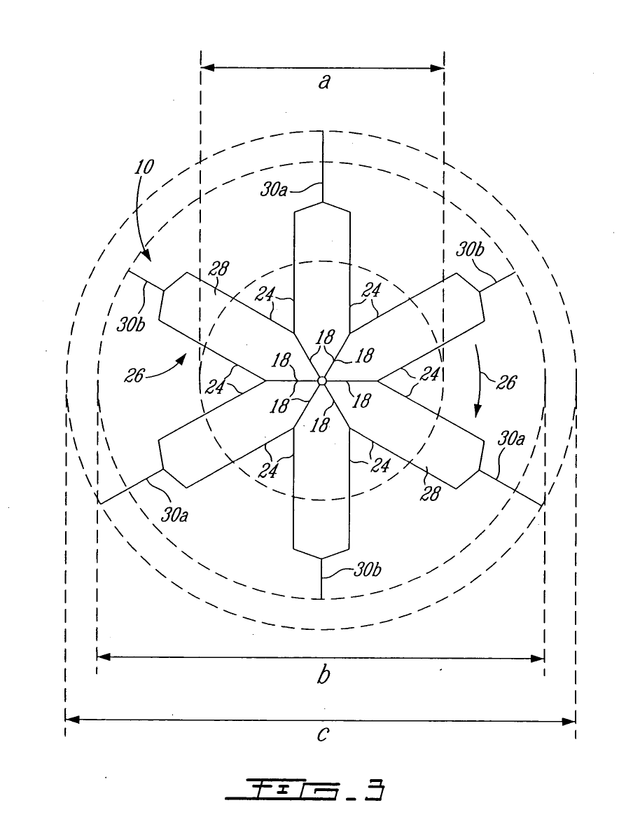

As best shown in FIG. 2, the filter 10 is provided at a proximal or downstream end thereof with a retrieval hook 15 extending from a central hub 16 from which radiates a set of primary branches 18 (6 branches in the illustrated embodiment). The primary branches 18 are provided in the form of fine wires or struts and are uniformly distributed in a wide opening cone configuration about a central longitudinal axis 20 of the filter 10 to define a relatively wide shoulder 22 immediately upstream of the central hub 16. According to an embodiment of the present invention, the shoulder has a diameter “a” of 15 mm (see FIG. 3). However, the diameter “a” could range from about 10 to 20 mm. The opening angle θ of the branches 18 is preferably of at least about 50 degrees when the filter 10 is in a fully expanded state. The opening angle “θ” will normally range from 50 to 70 degrees. As will be seen hereinafter, the fact that the primary branches 18 open rapidly close to the full width of the filter advantageously contributes to increase the amount of blood clots that can be trapped by the filter 10 in that it provides for the formation of a filter basket having added volume holding capacity as compared to fully conical filters. The relatively wide shoulder 22 also acts as an anti-tilting member by limiting the angular freedom of movement of the filter 10 in the blood vessel 12. This contributes to ensure that the filter 10 remains in axial alignment with the blood vessel 12 in which it has been implanted, thereby increasing the efficiency of the filter 10. This also facilitates retrieval of the filter by centering the retrieval hook 15.

As shown in FIGS. 1 to 3, each primary branch 18 bifurcates at a Y-shaped junction into a pair of diverging main branches 24 that extend angularly outwardly and then linearly downwardly away from the downstream end of the filter 10. The main branches 24 are collectively arranged in a generally cylindrical or slightly conical geometric configuration about the central axis 20 of the filter 10. The filter 10 is less likely to cause occlusion due to its cylindrical shape which can accommodate larger amounts of thrombus as compared to cone-shaped filters. According to one aspect of the present invention, each main branch 24 connects at a distal end thereof to an adjacent main branch 24 originating from a different primary branch 18, thereby providing a sturdy framework of branches interconnected to one another so that a force applied to a single branch will be automatically distributed to the other branches. The main branches 24 are grouped in pairs so as to form a set of circumferentially-spaced legs 26 (6 in the illustrated embodiment), each leg 26 being partly formed of two adjacent main branches 24 which are united together at their distal ends through a V-shaped junction but which are connected at their respective proximal ends to different primary branches 18. Each leg 26 defines an open cell 28 having opposed triangular ends and an intermediate rectangular section.

Each leg 26 terminates into an anchoring limb 30 extending in continuity from the lower V-shaped junction of two adjacent main branches 24. Each anchoring limb 30 has a hooked distal end 32 for penetrating the wall of the blood vessel 12 to anchor the filter 10 against movement. As shown in FIG. 3, the limbs 30 are preferably angled outwardly by an angle β ranging from 120 to 160°. The bend in each limb 30 contributes to minimize the surface of contact between the legs 26 and the wall of the blood vessel 12. By so limiting the surface of contact between the body of the filter 10 and the wall of the blood vessel 12, the growth of tissue over the filter 10 is significantly reduced and, thus, the filter 10 can remain implanted in the vessel 12 for a longer period of time without becoming securely embedded in the vessel wall. This advantageously enables easy retrieval of the filter 10 for extend periods of time.

To maintain the filter 10 centered in the vessel with minimal contact therewith, the limbs 30 are not all equal in length but rather includes a set of long limbs 30a and a set of short limbs 30b so as to provide two anchoring levels. By so anchoring the filter 10 at two longitudinally spaced-apart locations in the blood vessel 12, the filter 10 remains more stable even though it only has discrete points of contact with the vessel wall. This is also advantageous in that the pressure exerted by the filter 10 on the blood vessel are more uniformly distributed, thereby reducing the risk of local injury to the vessel wall.

As best seen from FIG. 3, the legs 26 are alternately provided with long and short anchoring limbs 30a and 30b to provide a succession of short and long anchoring limbs 30 about the open end of the filter basket. The distal hooked ends of the short anchoring limbs 30b lie on a circle having a diameter “b” ranging from 30 to 45 mm when the filter is fully expanded. Likewise, the distal hooked ends of the long anchoring limbs 30a lie on a circle having a diameter “c” ranging from 30 to 45 mm when the filter is fully expanded.

According to an embodiment of the present invention, the struts forming the primary branches 18 and the main branches 24 are rectangular in cross-section. For instance, each strut could have a cross-section of 0.25 mm by 0.35 mm. The anchoring limbs 30 could also have a rectangular cross-section but of 0.7 mm by 0.25 mm.

According to another feature of the present invention, there is provided a rapid transition in width from the limbs 30 to the fixation hooks 32 to prevent the limbs 30 from penetrating completely through the wall of the blood vessel.

1. A blood clot filter comprising a set of diverging primary branches extending from a common central hub, each primary branch bifurcating into a pair of main branches, wherein each main branch connects at a distal end to an adjacent main branch originating from a different primary branch to form a series of interconnected diverging anchoring members.

2. A blood clot filter as defined in claim 1, wherein there is provided “n” primary branches for “2n” main branches and “n” anchoring members.

3. A blood clot filter as defined in claim 1, wherein a retrieval aid projects from said central hub.

4. A blood clot filter as defined in claim 1, wherein the main branches define a generally cylindrical blood clot reservoir, and wherein said diverging anchoring members extend outwardly at an angle from said reservoir.

5. A blood clot filter as defined in claim 1, wherein said primary branches define a wide opening cone section merging into a generally cylindrical axially extending section delimited by the main branches.

6. A blood clot filter as defined in claim 1, wherein the main branches define a blood clot reservoir, and wherein every other anchoring member is longer than a preceding one to provide a succession of long and short interconnected anchoring members about an open upstream end of said reservoir.

7. A blood clot filter as defined in claim 6, wherein the long and short anchoring members are provided with hooked ends which lie on two different concentric circles.

8. A blood clot filter as defined in claim 1, wherein said primary branches are arranged to define a shoulder at a trailing end of the filter.

9. A blood clot filter as defined in claim 8, wherein said shoulder defines an angle θ of at least 50 degrees relative to a longitudinal axis of the filter.

10. A blood clot filter as defined in claim 8, wherein the main branches are bent at the level of said shoulder and then extend axially away from said shoulder to form a generally cylindrical blood clot receiving basket.

11. A blood clot filter as defined in claim 4, wherein the anchoring members are angled outwardly.

12. A blood clot filter as defined in claim 1, wherein the anchoring members are provided with respective hooked distal end portions which are angled outwardly by an angle β ranging from about 120 degrees to about 160 degrees.

13. A blood clot filter as defined in claim 1, wherein each primary branch bifurcates into a pair of main branches at a Y-shaped junction.

14. A filter positionable into a blood vessel for trapping blood clots, comprising a number of diverging legs extending from a common central hub to define a blood clot reservoir, wherein adjacent legs share a common root from which branches off at least two main branches, each main branch connecting at a distal end to an adjacent main branch to form a plurality of interconnected anchoring members about an open upstream end of said blood clot reservoir.

15. A filter as defined in claim 14, wherein each leg defines an open cell having opposed triangular ends and an intermediate elongated sections.

16. A filter as defined in claim 14, wherein there is provided “n” roots for “2n” main branches and “n” anchoring members.

17. A filter as defined in claim 14, wherein a retrieval hook aid projects from said central hub.

18. A filter as defined in claim 14, wherein said anchoring members extend outwardly at an angle β from a central axis of said blood clot reservoir.

19. A filter as defined in claim 14, wherein said roots define a wide opening cone section merging into a generally cylindrical axially extending section delimited by the main branches.

20. A filter as defined in claim 14, wherein every other anchoring member is longer than a preceding one to provide a succession of long and short interconnected anchoring members about the open upstream end of said reservoir.

21. A filter as defined in claim 20, wherein the long and short anchoring members are provided with hooked ends which lie on two different concentric circles.

22. A filter as defined in claim 14, wherein said roots are arranged to define a shoulder at a trailing end of the filter.

23. A filter as defined in claim 22, wherein said shoulder defines an angle θ of at least 50 degrees relative to a longitudinal axis of the filter.

24. A filter as defined in claim 22, wherein the main branches are bent at the level of said shoulder and then extend axially away from said shoulder.

25. A filter as defined in claim 14, wherein the anchoring members are angled outwardly.

26. A filter as defined in claim 14, wherein the anchoring members are provided with respective hooked distal end portions which are angled outwardly by an angle β ranging from about 120 degrees to about 160 degrees.

27. A filter positionable into a blood vessel for filtering blood clots from a stream of blood, the filter comprising a central hub, a plurality of interconnected legs extending from said central hub and arranged about a central longitudinal axis of the filter to form a filter basket having an open end, wherein each leg is provided at a distal end thereof opposite said hub with an anchoring limb adapted to anchor the filter basket to the blood vessel, wherein said anchoring limbs are distributed about said open end of said filter basket and extend at different distances from said central hub to provide at least two anchoring levels.

28. A filter as defined in claim 27, wherein said filter basket has a generally cylindrical configuration.

29. A filter as defined in claim 27, wherein each leg is provided with a pair of struts having respective distal ends branching to said anchoring limb of said leg.

30. A filter as defined in claim 27, wherein said filter basket has a generally conical end portion merging into a generally cylindrical portion.

31. A filter as defined in claim 27, wherein adjacent legs share a common root from which branches off at least two main branches, each main branch connecting at a distal end to an adjacent main branch before merging into said anchoring limbs.

32. A filter as defined in claim 27, wherein each leg defines an open cell having opposed triangular ends and an intermediate elongated sections.

33. A filter as defined in claim 31, wherein there is provided “n” roots for “2n” main branches and “n” anchoring members.

34. A filter as defined in claim 31, wherein every other anchoring limb is longer than a preceding one to provide a succession of long and short interconnected anchoring limbs.

35. A filter as defined in claim 34, wherein the long and short anchoring limbs are provided with hooked ends which lie on two different concentric circles.

36. A filter as defined in claim 31, wherein said roots are arranged to define a shoulder at a trailing end of the filter.

37. A filter as defined in claim 36, wherein said shoulder defines an angle θ of at least 50 degrees relative to a longitudinal axis of the filter.

38. A filter as defined in claim 36, wherein the main branches are bent at the level of said shoulder and then extend axially away from said shoulder.

39. A filter as defined in claim 27, wherein the anchoring limbs are provided with respective hooked distal end portions which are angled outwardly by an angle β ranging from about 120 degrees to about 160 degrees.
